(19) United States
(12) Patent Application Publication
(10) Pub. No.: US 2018/0271913 A1
(43) Pub. Date: Sep. 27, 2018

(54) PHARMACEUTICAL COMPOSITIONS FOR PROMOTING HAIR FOLLICLE REGENERATION AND METHODS FOR PREPARING THE SAME

(71) Applicants: NATIONAL CHENG KUNG UNIVERSITY, TAINAN CITY (TW); NATIONAL CHENG KUNG UNIVERSITY HOSPITAL, TAINAN CITY (TW)

(72) Inventors: CHIA-CHING WU, TAINAN CITY (TW); CHIN-FU HUANG, TAINAN CITY (TW); YA-JU CHANG, TAINAN CITY (TW); YUAN-YU HSUEH, TAINAN CITY (TW)

(21) Appl. No.: 15/464,648

(22) Filed: Mar. 21, 2017

(51) Int. Cl. A61K 35/36; C12N 5/071; C12N 5/00; A61K 35/28

(52) U.S. Cl. CPC A61K 35/36; C12N 5/0629; C12N 2502/1382; A61K 35/28; C12N 2533/72; C12N 5/0012

(57) ABSTRACT

Pharmaceutical compositions for promoting hair follicle regeneration and their respective preparation methods are provided to overcome the ineffectiveness and low efficiency of the conventional hair follicle regeneration techniques. The methods for preparing the pharmaceutical compositions for promoting hair follicle regeneration include seeding dermal papilla cells and adipocyte lineage cells on a culture material in order for the dermal papilla cells and the adipocyte lineage cells to jointly assemble cell spheres. The pharmaceutical compositions can effectively enhance the effect and efficiency of hair follicle regeneration and thereby promote the regeneration of hair follicles.

### BACKGROUND OF THE INVENTION

### 1. Technical Field

The present invention relates to the regeneration of hair follicles and more particularly to pharmaceutical compositions for promoting hair follicle regeneration and their respective preparation methods, wherein dermal papilla cells and adipocyte lineage cells jointly form cell spheres to promote the regeneration of hair follicles.

### 2. Description of Related Art

Loss of hair can be attributed to a variety of factors, including for example the effects of a micro- or macro-environment on hair follicles, and both the medical and the academic communities have paid much attention to effective suppression of hair loss and regeneration of hair follicles. Nowadays, the two mainstream treatments for hair loss are medication and hair transplantation. Medication serves mainly to prevent further hair loss but is unable to regrow the lost hair. Hair transplantation, on the other hand, involves transplanting healthy hair follicles to a hair-losing area but is subject to the obtainment of a sufficient quantity of such follicles, for the severer the hair loss, the more the healthy hair follicles to be transplanted. According to the latest scientific research, cells related to inducing hair growth include dermal papilla cells (DP cells), dermal sheath cells, skin-derived precursor cells, and mesenchymal stem cells (MSCs). In particular, the aggregation of DP cells is known to be crucial to follicular regeneration (M. Ohyama et al., 2013), and once cultured DP cells are aggregated to form spheres, their ability to induce hair follicle regeneration is enhanced (B. M. Kang et al., 2012).

Studies also show that adipose tissues around a hair follicle play an important role in promoting hair follicle growth (L. S. Hansen et al., 1984). According to further studies, proliferation of DP cells can be significantly increased by culturing the DP cells in a conditioned medium (CM) obtained from culture of adipose-derived stem cells (ASCs). This implies that the growth factors secreted by the ASCs during culture can advance the growth of DP cells indirectly (Peipei Zhang et al., 2014). All the foregoing studies on hair follicle regeneration, however, are in the research stage and have a long way to go before they are industrially applicable.

In light of the above, the inventor of the present invention gathered related information and incorporated years of practical experience in the industry—particularly the technical content of Taiwan Invention Patent No. 14903375, which was granted to the inventor of the present invention and discloses co-culture of DP cells and adipocyte lineage cells by a method of enabling cells to automatically form spheres during culture under the action of chitosan—into repeated trials and modifications in order to create inventions capable of effectively promoting hair follicle regeneration.

### BRIEF SUMMARY OF THE INVENTION

Pharmaceutical compositions for promoting hair follicle regeneration and their respective preparation methods are provided to address the ineffectiveness and low efficiency of the conventional techniques of promoting hair follicle regeneration with cell spheres formed by dermal papilla cells (DP cells). In essence, DP cells are used in conjunction with adipocyte lineage cells to form cell spheres which are more effective in promoting hair follicle regeneration than their prior art counterparts.

The present invention provides a method for preparing a pharmaceutical composition for promoting hair follicle regeneration, wherein the method includes the steps of:

(a): seeding DP cells on a culture material in order for the DP cells to aggregate a sphere automatically; and (b): seeding adipocyte lineage cells on the culture material in order for the adipocyte lineage cells to enclose the sphere and thereby form a core-shell sphere including at least two layers of different cells.

The present invention provides another method for preparing a pharmaceutical composition for promoting hair follicle regeneration, wherein the method includes the step of seeding DP cells and adipocyte lineage cells on a culture material in order for the DP cells and the adipocyte lineage cells to jointly assemble a mixed sphere.

Preferably, the adipocyte lineage cells are human adipocytes, human adipose-derived stem cells (ASCs), or a combination of the above.

Preferably, the culture material includes a base and a chitosan film, wherein the chitosan film is formed by coating the base with a 1% w/v chitosan solution.

Preferably, the DP cells and the adipocyte lineage cells are seeded in the ratio of 2 to 1.

The present invention also provides a pharmaceutical composition for promoting hair follicle regeneration in which the pharmaceutical composition includes the core-shell sphere prepared by the first method stated above for preparing a pharmaceutical composition for promoting hair follicle regeneration.

The present invention further provides a pharmaceutical composition for promoting hair follicle regeneration in which the pharmaceutical composition includes the mixed sphere prepared by the second method stated above for preparing a pharmaceutical composition for promoting hair follicle regeneration.

The Effects of the Present Invention Are:

1. By co-culturing DP cells and adipocyte lineage cells to assemble cell spheres, not only are the DP cells rendered more effective in inducing hair follicle regeneration, but also the growth factors secreted by the adipocyte lineage cells (i.e., paracrine secretion) stimulate proliferation of the DP cells to increase cell survival rate.

2. As the desired cell spheres can be obtained in large quantities by in-vitro cell culture and applied to seriously hair-losing skin to encourage regeneration of hair follicles, the present invention has overcome the aforesaid problem of conventional hair transplantation, namely the unavailability of an unlimited quantity of healthy hair follicles.

### BRIEF DESCRIPTION OF THE SEVERAL VIEWS OF THE DRAWINGS

FIG. 1 are graphs showing how each of five seeding quantities of DP cells in the present invention affects the DP cells' inducing ability;

FIG. 2 shows the fluorescently tagged structures of core-shell spheres and mixed spheres in the present invention;

FIG. 3A shows adipose-derived stem cells (ASCs) in the present invention and adipocytes differentiated therefrom, before and after they are stained with oil-red O;

FIG. 3B shows the qualitative test results of ASCs in the present invention and of adipocytes differentiated therefrom

FIG. 3C shows immunofluorescence images of core-shell spheres and mixed spheres in the present invention;

FIG. 4A shows skin issues before and after they are stained with alkaline phosphatase according to the present invention;

FIG. 4B shows the number of hairs grown from patch assay in the present invention;

FIG. 4C shows skin tissues stained with hematoxylin and eosin according to the present invention; and

FIG. 4D shows immunohistochemistry images of skin tissues according to the present invention.

### DETAILED DESCRIPTION OF THE INVENTION

To elucidate the features and advantages of the pharmaceutical compositions of the present invention for promoting hair follicle regeneration and the methods for preparing the same, a detailed description of some preferred embodiments of the invention is given below with reference to the accompanying drawings.

The present invention provides a method for preparing a pharmaceutical composition for promoting hair follicle regeneration, wherein the method includes: step (a) of seeding dermal papilla cells (DP cells) on a culture material in order for the DP cells to aggregate a sphere automatically; and step (b) of seeding adipocyte lineage cells on the culture material in order for the adipocyte lineage cells to enclose the sphere and thereby form a core-shell sphere including at least two layers of different cells.

As a person of ordinary skill in the art would understand, the term “adipocyte lineage cells” refers to two major types of cells in adipose tissues, namely mature adipocytes and stromal vascular cells, the latter of which consist essentially of adipocyte precursor cells, i.e., the so-called adipose-derived stem cells (ASCs). In the embodiments disclosed herein, mature adipocytes and ASCs of human origin are used by way example.

The present invention further provides a method for preparing a pharmaceutical composition for promoting hair follicle regeneration wherein the method includes the step of seeding DP cells and adipocyte lineage cells on a culture material in order for the DP cells and the adipocyte lineage cells to jointly assemble a mixed sphere.

Preferably, the adipocyte lineage cells are human adipocytes, human ASCs, or a combination of the above.

Preferably, the culture material includes a base and a chitosan film, and the chitosan film is formed by coating, the base with a 1% w/v chitosan solution. In the embodiments disclosed herein, the base is a commercially available, tissue culture plate (TCP), or better known as a culture dish. To enable cell culture, it is understood that the culture medium required by the cells to be cultured is added into the culture dish.

Preferably, the DP cells and the adipocyte lineage cells are seeded in the ratio of 2 to 1.

The present invention also provides a pharmaceutical composition for promoting hair follicle regeneration, wherein the pharmaceutical composition includes DP cells aggregating a sphere and adipocyte lineage cells enclosing the sphere. The adipocyte lineage cells are human adipocytes, human ASCs, or a combination of the above. The DP cells and the adipocyte lineage cells are in the ratio of 2 to 1.

The present invention further provides a pharmaceutical composition for promoting hair follicle regeneration wherein the pharmaceutical composition includes DP cells and adipocyte lineage cells, and wherein the DP cells and the adipocyte lineage cells jointly assemble a mixed sphere. The adipocyte lineage cells are human adipocytes, human ASCs, or a combination of the above. The DP cells and the adipocyte lineage cells are in the ratio of 2 to 1.

Hereinafter, implementation of the present invention is described with reference to some preferred embodiments of the invention. The description should be able to enable a person skilled in the art to readily understand other advantages and effects of the invention. The invention may also be implemented or applied in ways other than illustrated by the embodiments; in other words, the present invention can be modified or altered in many ways without departing from the spirit of the invention.

In the embodiments that follow, the data of each sample and each time point are shown as means of triplicate values with positive and negative standard deviations. All the values were analyzed by one-way analysis of variance (one-way ANOVA), with a p-value less than 0.05 indicating a significant value.

### Embodiment 1: Separation and In-Vitro Culture of Cells

The human adipocyte lineage cells used in this experiment were obtained from volunteer patients in National Cheng Kung University Hospital with the approval of the Internal Review Board (IRB). More specifically, adipose tissues obtained from liposuction procedures were separated and cultured to obtain the human ASCs needed for the experiment. The stem cells were cultured in a Dulbecco's, modified Eagle's medium (DMEM) containing 10% fetal bovine serum (FBS) and 1% penicillin/streptomycin (P/S) under the conditions of 37° C. and 5% carbon dioxide. To ensure satisfactory differentiation ability, all the human ASCs used in the experiment were of generations earlier than the 10th generation. The process flows/methods of cell separation and cell culture in this experiment were based on the techniques previously established in the inventor's laboratory (Y. Y. Hsueh et al., 2012, 2014). Since the process flows/methods are comprehensible to a person of ordinary skill in the art and do not constitute an essential part of the invention, they will not be detailed herein.

The human ASCs obtained were also cultured in a culture medium capable of inducing differentiation into adipocytes in order to produce mature adipocytes, the objective being to compare the hair follicle regeneration inducing abilities of cell spheres formed by co-culturing ASCs and DP cells and of those formed by co-culturing adipocytes and DP cells. In this experiment, the culture medium capable of inducing differentiation into adipocytes was prepared by adding the following chemicals: 0.5 mM 3-isobutyl-l-methylxanthine (IBMX, Sigma), 1M dexamethasone (Sigma), 10 g/ml insulin (Sigma), and 1M indomethacin (Sigma). As the inducing method is well-known to a person of ordinary skill in the art, a detailed description of the method is omitted herein.

All the animal used in the experiment were provided by the Animal Center at National Cheng Kung University with the approval of experimental procedures from the Institutional Animal Care and Use Committee (IACUC). The DP cells were taken from vibrissal hair follicle of 6 to 8-week-old SD rats. The hair follicle taken were put in a 2.5 mg/ml type-1 collagenase solution for digestion at 37° C. for 3 hours and then centrifuged at 800 r.p.m. for 10 minutes to obtain cell pellets. The cell pellets were gradually dispersed with a DMEM after the supernatant was removed, and DP cell clusters were sorted out under a microscope and transferred to a culture flask, where the clusters were cultured in a DMEM containing 20% FBS (Hyclone) and 1% P/S (Invitrogen) for 5 days. Once the DP cell clusters expanded outward, they were washed with phosphate-buffered saline (PBS) once and then reacted with a 0.5% trypsin-EDTA (Invitrogen) at 37° C. for a proper amount of time, before DP cells were extracted for subculturing in a DMEM containing 10% FBS (Hyclone) and 1% P/S. Subsequent subcultures were carried out in the same way. It should be pointed out that the DP cells used in the experiment were obtained from SD rats by way of example only (because of their relative ease of availability) and may be obtained from other sources as well. Based on the spirit of the present invention, human DP cells can be used in the same experiment to achieve the same results.

In addition, keratinocytes (KCs) were obtained by separating them from the skin of one-day-old mice (C57BL/6) in the following manner To begin with, mouse skin was obtained and cut into small pieces, which were put into a Hank's balanced salt solution (HBSS, Invitrogen) containing 0.5% dispase (Invitrogen) and were shook at 4° C. overnight for digestion. After that, the epidermis was separated from the dermis, washed with an HBSS, cut into fine pieces, digested with 0.1% trypsin at 37° C. for 20 minutes. Once digestion was inactivated by adding a DMEM, was and then filtered through a 70 μm cell strainer (BD, USA), and the filtrate was centrifuged at 1200 r.p.m. for 5 minutes. If the culture of KCs was required, they were cultured on collagen-coated dish (collage type I, BD) with keratinocyte basal medium(Cell applications, USA). The precipitated keratinocytes were collected for use in the subsequent in-vivo hair follicle regeneration experiment. Keratinocytes were used in the in-vivo hair follicle regeneration experiment because they are essential to, and will interact with DP cells to induce hair regeneration (A. Osada et al., 2007).

### Embodiment 2: Preparation of Chitosan-Coated Culture Plates and Assembling of Cell Spheres

In this experiment, cells were cultured in a tissue culture plate coated with chitosan such that cells which would otherwise attaching the plate surface during growth were prevented from doing so. Instead, cells grew by forming spheroid suspended in the culture medium. The coated tissue culture plate was prepared as follows. 1% w/v chitosan powder (85% deacetylation, Sigma-Aldrich) was dissolved in 1 M acetic acid. The solution was stirred for 24 hours and then filtered twice to remove impurities and produce a chitosan coating solution. A 10-cm tissue culture plate was coated with 10 ml of the chitosan coating solution, or each well of a 96-well culture plate, with 200 μl of the chitosan coating solution. In either case, the coated plate was oven-dried at 70° C. for 24 hours, and a chitosan film was formed as a result. The chitosan film was neutralized by applying aqueous 1 N NaOH for 30 min and then washed with distilled water, and then sterilized by exposure to ultraviolet light for 12 hours. Thus, the chitosan-coated culture plate for use in this embodiment was obtained. In order for the cultured cells to form spheres, cells were seeded at an appropriate density (i.e., a proper quantity of cells were seeded) in the chitosan-coated and culture medium-containing culture plate, which was subsequently placed into a cell culture incubator and used a shaker with 130 r.p.m. and 37° C. for 3 days to produce aggregated cell spheres.

### Embodiment 3: Culture Condition Test for Cell Spheres

3.1 This experiment was designed to determine the optimal quantity of cells to be seeded, or more particularly the optimal quantities of DP cells and adipocyte lineage cells to be seeded for co-culture. Different quantities of DP cells were seeded on 96-well culture plates in order to be cultured (i.e., to aggregate spheres), and the expression of proteins (e.g., HEY1 and versican) associated with the induction of hair follicle regeneration was then assessed. In this experiment, separate chitosan-coated plates were seeded with 0.5×10⁴, 1×10⁴, 2×10⁴, 5×10⁴, and 1×10⁵ cells respectively, added with a DMEM containing 10% FBS and 1% P/S, and then cultured in the foregoing manner (i.e., shook at 130 r.p.m. and 37°C. for 3 days). Once harvested, the cell spheres were stained for immunofluorescence to compare the expression intensities of protein, markers HEY1 (1:250, Thermo), versican (VSAN, 1:100, Santa Cruz), and vimentin (VIM, 1:250, Abcam). The immunofluorescence staining technique employed in the experiment is a common protein marker expression analysis method comprehensible to all those of ordinary skill in the art and was used in conjunction with an inverted microscope (or multiphoton confocal microscope) and fluorescence analysis software to enable quantitative comparison. As immunofluorescence is not an essential technical feature of the present invention, a detailed description of its process is omitted herein.

3.2 Experiment Results

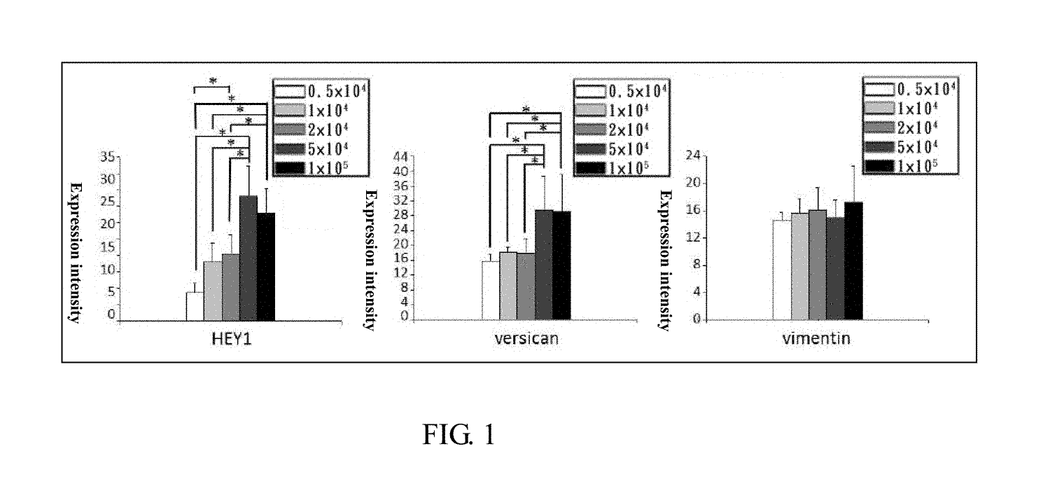

In FIG. 1, which shows the results of this experiment, each horizontal axis represents the five quantities of cells seeded, and each vertical axis, the relative expression intensity of a certain protein marker. The experiment results show that when 5×10⁴ and 1×10⁵ cells were seeded per well, the protein markers HEY1 and versican had relatively high expression intensities. In other words, cell spheres obtained by seeding 5×10⁴ to 1×10⁵ cells were better at promoting hair follicle regeneration. The optimal result was obtained when 5×10⁴ cells were seeded. On the other hand, better results were achieved when 2.5×10⁴ adipocyte lineage cells were seeded per well for co-culture with DP cells. Therefore, DP cells and adipocyte lineage cells are preferably seeded in the ratio of 2 to 1. Vimentin, which served as a reference of comparison, is a mesenchymal marker used only to show that the quantity of seeded cells has no impact on its protein expression.

### Embodiment 4: Formation of Cell Spheres by Co-Culture of DP Cells and ASCs

4.1 Formation of Core-Shell Spheres

This experiment provides a method for forming core-shell spheres by co-culturing DP cells and human ASCs. The method, which involves a sequential cell seeding technique, includes the steps of: seeding 5×10⁴ DP cells on a chitosan-coated 96-well culture plate (as prepared in embodiment 2) with a DMEM containing 10% FBS and 1% P/S, culturing for 48 hours to form the cores of core-shell spheres, and then adding 2.5×10⁴ human ASCs for co-culture so that the ASCs gather around and enclose each sphere formed by the DP cells, forming core-shell spheres each containing at least two layers, namely a core and a shell. Please note that the two-layer cell sphere structure is only exemplary. Based on the spirit of the present invention, different cells may be added sequentially for co-culture to form cell spheres of more than two layers.

4.2 Regeneration of Fixed Spheres

This experiment provides a method for forming mixed spheres by co-culturing DP cells and human ASCs. This method includes the steps of: mixing 5×10⁴ DP cells with 2.5×10⁴ human ASCs sufficiently, seeding the mixed cells on a chitosan-coated 96-well culture plate (as prepared in embodiment 2) with DMEM containing 10% FBS and 1% P/S, and co-culturing for 3 days to assemble mixed spheres.

4.3 Verification of the Structures of the Core-Shell Spheres and Mixed Spheres Obtained

The structures of the core-shell spheres and mixed spheres obtained were verified by immunofluorescence staining. Before seeding, the ASCs were tagged by treatment with DiI (a red fluorescent cell stain, Invitrogen) for 30 minutes, in order for the DiI stain to bind to the cell membranes of the ASCs to facilitate tracking. Apart from that, antibodies to the protein markers HEY 1 and versican (VCAN) (both associated with the induction of hair follicle regeneration) of the DP cells were tagged with a green fluorescent stain to enable observation of protein expression. The analysis was conducted using a confocal microscope (either a spinning-disc confocal microscope (DSU IX81, Olympus) or a multiphoton confocal microscope (FV1000MPE, Olympus)) together with fluorescence analysis software (ImageJ software).

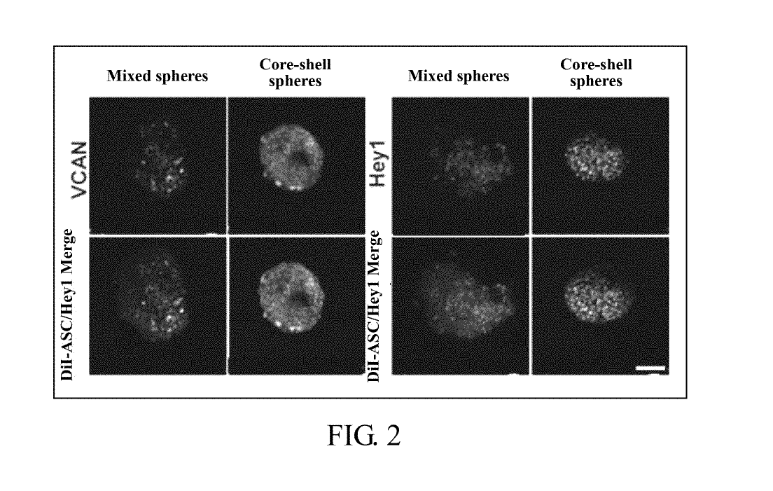

FIG. 2 shows the fluorescently tagged structures of the core-shell spheres and mixed spheres in the present invention, with the size bar being 100 μm. As shown in the images of FIG. 2, DP cells (or its VCAN and HEY 1, which were tagged to emit green fluorescence) and ASCs (or the DiI stain bound thereto to emit red fluorescence) did exist in the mixed spheres, and both types of cells distributed themselves evenly within the spherical spaces. In the core-shell spheres, DP cells (or its VCAN and HEY1, which were tagged to emit green fluorescence) and ASCs (or the DiI stain bound thereto to emit red fluorescence) also existed but formed a two-layer core-shell structure, in which the core was composed of the DP cells and the shell was composed of of the ASCs. It is thus verified that the methods provided by the present invention for preparing cell spheres are viable.

### Embodiment 5: Cell Spheres Formed by Co-Culture of DP Cells and ASCs vs. Cell Spheres Formed by Co-Culture of DP Cells and Adipocytes Differentiated from ASCs

5.1 Induced Differentiation from ASCs to Adipocytes

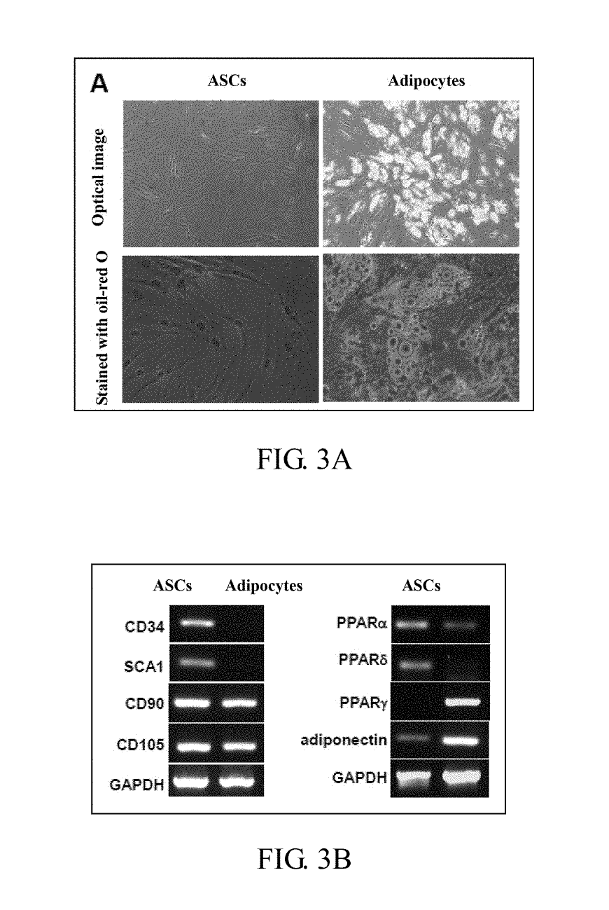

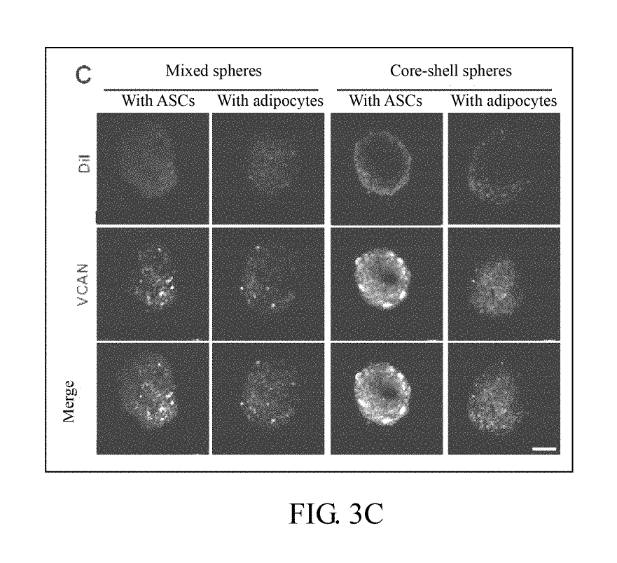

The ASCs obtained from embodiment 1 were induced to differentiate into adipocytes. FIG. 3A shows microscopic images of the ASCs and the adipocytes differentiated therefrom, before and after the cells were stained with oil-red O. According to the images, only the adipocytes differentiated from the ASCs were stained red by oil-red O and are thus verified as mature adipocytes. FIG. 3B shows the results of applying reverse transcription-polymerase chain reaction (RT-PCR) to analyzing the gene expression of stem cell markers, such as CD34 and SCA1, and adipocyte-specific markers, such as the peroxisome proliferator-activated receptor γ (PPARγ) and adiponectin. RT-PCR is a well-known gene expression analysis method and therefore will not be described herein. For a detailed description of the method/process flow of RT-PCR, reference can be made to technical data previously established by the inventor of the present invention (Y. Y. Hsueh et al., 2012). The analysis results show that the ASCs kept the expressions of the CD34 and SCA1 genes while the adipocytes no longer expressed CD34 or SCA1, meaning the adipocytes no longer had the features of stem cells. The GAPDH gene shown in FIG. 3B was used as the housekeeping gene. The analysis results also show that the adipocytes expressed PPARγ and adiponectin significantly, which proves that the ASCs in the experiment were successfully induced to differentiate into adipocytes.

The primer sequences used in this experiment are tabulated as follows:

| | | Product |
|---|---|---|
| | | size |
| Gene | Primer sequence (5′ to 3′) | (bp) |
| CD34 | Forward: GTGTTTGCTGATGGTCTTGG | 340 |
| | Reverse: ATTGGCCTTTCCCTGAGTCT | |
| SCA1 | Forward: AGCTCTTTGATCTGCCGTGT | 357 |
| | Reverse: CCGACTTCTCCAGTTTACGG | |
| PPARγ | Forward: TGGGGATGTCTCACAATGC | 346 |
| | Reverse: CGAAACTGGCACCCTTGA | |
| adiponectin | Forward: CAGGAGATGCTGGAATGACA | 325 |
| | Reverse: TGGTCGTAGGTGAAGAGAACG | |
| GAPDH | Forward: TCTCTGCTCCTCCCTGTTCTA | 400 |
| | Reverse: GGCGGAGATGATGACCCTTT | |

5.2 The Effects of Cell Spheres Formed by Co-Culturing DP Cells with ASCs or with Adipocytes Differentiated from ASCs on the Expression of Versican (WAN)

This experiment was intended to determine how cell spheres formed by co-culturing DP cells with adipocytes, rather than ASCs, affect the way the DP cells express the protein marker VCAN. In this experiment, adipocytes and DP cells were co-cultured to form two types of cell spheres (both core-shell spheres and mixed spheres) in the same way as described above for co-culturing DP cells and ASCs to form cell spheres, so a detailed description of the process will not be repeated here. Like the ASCs, the adipocytes were tagged by treatment with DiI for 30 minutes before seeding, in order to facilitate tracking, and after the DP cells forming the cell spheres, particularly the protein VCAN of the DP cells, was subjected to immunofluorescence staining with a green fluorescent stain. Observation was made under the aforesaid confocal microscope. It can be seen in FIG. 3C that both the core-shell spheres and mixed spheres formed by co-culture of adipocytes and DP cells expressed the protein VCAN, though at a lower intensity than those formed by co-culture of ASCs and DP cells. This means that the better mode is to form cell spheres by co-culturing ASCs and DP cells. It is understood, however, that ASCs can be mixed with adipocytes and then co-cultured with DP cells to form cell spheres without departing from the spirit of the present invention.

### Embodiment 6: Patch Assay

In this experiment, each of five different types of cell combinations (1. DP cells; 2. DP cell spheres; 3. DP cell spheres+ASCs; 4. mixed spheres; and 5. core-shell spheres) was added with 2.5×10⁴ keratinocytes, then injected subcutaneously into male athymic nude mice, and allowed to live in vivo. After four weeks, full-thickness skin was surgically cut from the injected area of each mouse and subjected to tests such as alkaline phosphatase staining, hematoxylin and eosin (H&E) staining, and immunohistochemistry (IHC) staining, with a view to comparing the regeneration of hair follicles in the five types of cell combinations. Since alkaline phosphatase staining, H&E staining, and IHC staining are common staining analysis techniques comprehensible to a person of ordinary skill in the art, their process flows and steps will not be described herein.

6.1 Alkaline Phosphatase Staining

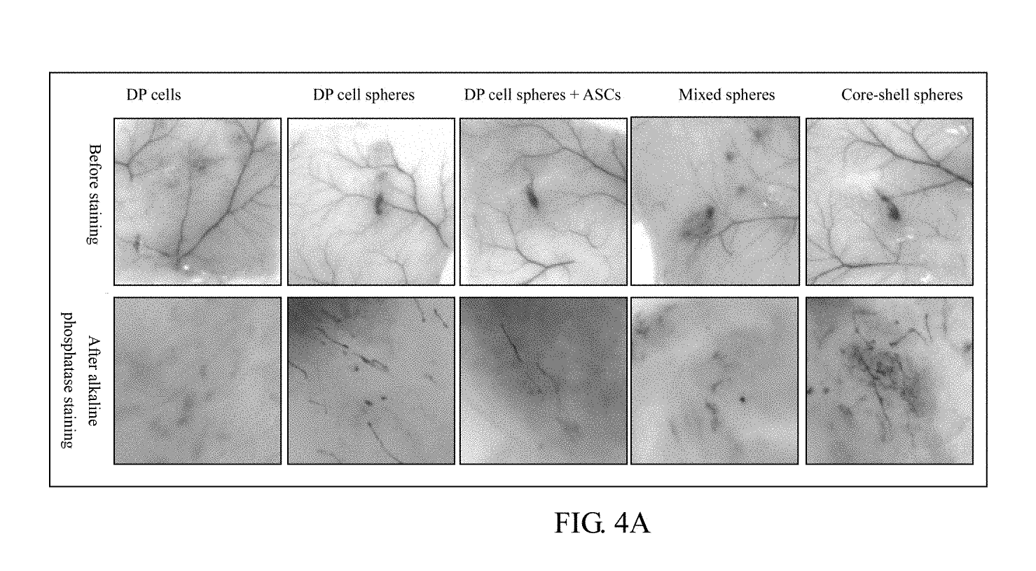

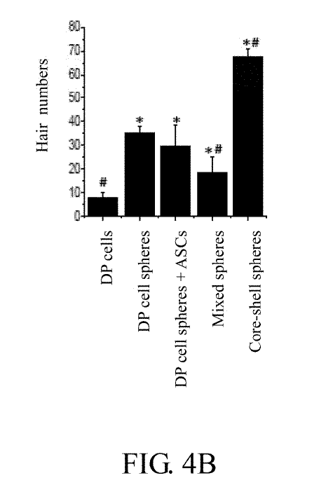

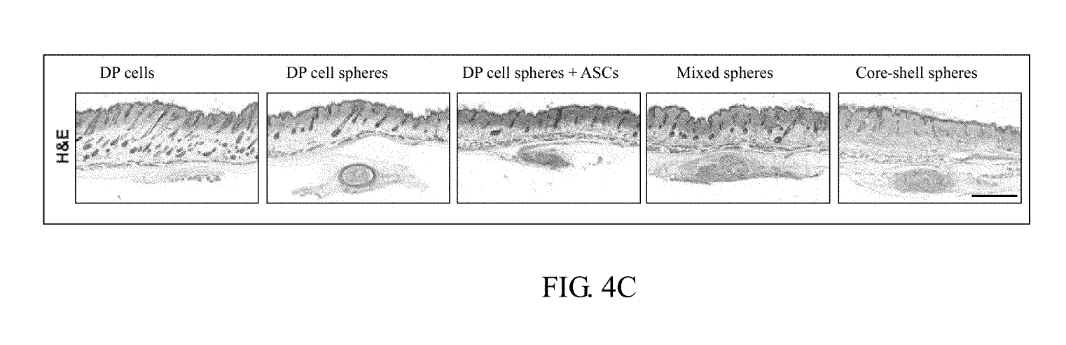

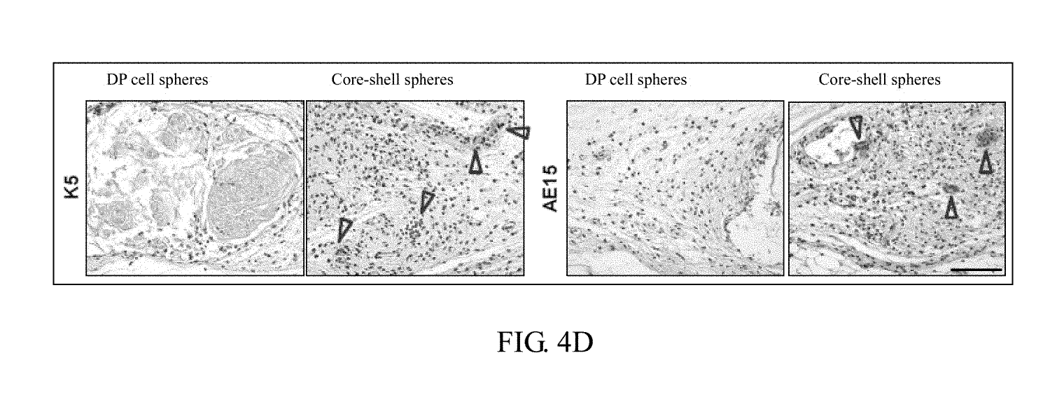

Alkaline phosphatase is a common indicator of the hair follicle regeneration inducing ability of DP cells. In this experiment, tests were conducted using a commercially available test kit (FAST BCIP/NBT (5-bromo-4-chloro-3-indolyl phosphate/nitro blue tetrazolium), Sigma), and the test operation conformed to the teachings of the user manual. As the test operation is not an essential technical feature of the present invention, a detailed description thereof is omitted herein. Hair images were taken by an optical microscope capable of photography. Referring to FIG. 4A, which shows photographs before and after alkaline phosphatase staining, the core-shell sphere group not only exhibits more pronounced pigmentation of hair follicles than the other four groups before alkaline phosphatase staining, but also has a more distinct brown stain than the other four groups after staining. Moreover, according to FIG. 4B, which shows the hair counting result obtained via a tissue scanning microscope (BX51, Olympus), the core-shell sphere group grew the largest number of hairs. It is thus verified that the core-shell spheres formed by co-culture of ASCs and DP cells had a notable ability to promote hair follicle regeneration. In other words, a pharmaceutical composition for promoting hair follicle regeneration preferably includes such core-shell spheres. It is also observed that all the groups 2-4 were more effective in promoting hair follicle regeneration than the DP cell group (group 1).

6.2 H&E Staining

H&E staining made it possible to observe formation of subcutaneous hair structures in each of the five groups. Referring to FIG. 4C, in which the size bar is 500 μm, the DP cell group (group 1) has only a subcutaneous cyst and no hair, whereas the other four groups show hair structures formation of various degrees. In particular, a significant formation of hair structures can be observed in the core-shell sphere group (group 5).

6.3 IHC Staining

This experiment further examined whether the hair structures induced from the core-shell sphere group (group 5) included structures that a normal hair follicle should have. The hair follicle structure-specific antibodies used are keratin 5 (K5. 1:200, Abcam) for staining the outer root sheath of a hair follicle and trichohyalin (AE15, 1:200 Abcam) for staining the inner root sheath and medulla of a hair follicle. The immunohistochemistry staining technique employed in this experiment is a widely used tissue staining analysis method which works on the following principle. To begin with, primary antibodies bind to specific protein antigens in a tissue, and then secondary antibodies with a signal bind to the primary antibodies. Signal detection is performed after the unbound secondary antibodies are washed away. The detection result will be positive if the primary antibodies have bound specifically to a certain region of the tissue. The details of the foregoing process are omitted herein.

FIG. 4D, in which the size bar is 100 μm, shows how the DP cell sphere group (group 2) and the core-shell sphere group (group 5) expressed keratin 5 (K5) and trichohyalin (AE15) after IHC staining. According to the images, only the core-shell sphere group (group 5) has dark brown colored areas (indicated by the arrows) corresponding respectively to K5 and AE15. This not only means that the hair follicles induced by the core-shell spheres formed by co-culture of ASCs and DP cells were complete in structure, but also confirms that the core-shell spheres formed according to the present invention by co-culture of ASCs and DP cells were indeed effective in promoting hair follicle regeneration.

According to the present invention as described above, DP cells and adipocyte lineage cells (especially ASCs) are either mixed in advance or seeded sequentially before they are co-cultured to form mixed spheres or core-shell spheres capable of inducing hair follicle regeneration, wherein the core-shell spheres are the more, effective inducer. The invention thus enables the preparation of large quantities of pharmaceutical compositions for promoting hair follicle regeneration.

While the present invention has been disclosed by way of the foregoing embodiments, it is understood that the embodiments are not intended to be restrictive of the scope of the invention, and, that minor changes and modifications are conceivable by a person skilled in the art without departing from the spirit and scope of the invention. The scope of patent protection sought by the applicant is defined by the appended claims.

What is claimed is:

1. A method for preparing a pharmaceutical composition for promoting hair follicle regeneration, comprising the steps of:
(a) seeding dermal papilla cells (DP cells) on a culture material in order for the DP cells to aggregate a sphere automatically; and
(b) seeding adipocyte lineage cells on the culture material in order for the adipocyte lineage cells to enclose the sphere and thereby form a core-shell sphere including at least two layers of different cells.

2. The method of claim 1, wherein the adipocyte lineage cells are human adipocytes, human adipose-derived stem cells (ASCs), or a combination thereof.

3. The method of claim 1, wherein the culture material includes a base and a chitosan film, and the chitosan film is formed by coating the base with a 1% w/v chitosan solution.

4. The method of claim 1, wherein the DP cells and the adipocyte lineage cells are seeded in a ratio of 2 to 1.

5. A method for preparing a pharmaceutical composition for promoting hair follicle regeneration, comprising the step of seeding dermal papilla cells (DP cells) and adipocyte lineage cells on a culture material in order for the DP cells and the adipocyte lineage cells to jointly assemble a mixed sphere.

6. The method of claim 5, wherein the adipocyte lineage cells are human adipocytes, human adipose-derived stem cells (ASCs), or a combination thereof.

7. The method of claim 5, wherein the culture material includes a base and a chitosan film, and the chitosan film is formed by coating the base with a 1% w/v chitosan solution.

8. The method of claim 5, wherein the DP cells and the adipocyte lineage cells are seeded in a ratio of 2 to 1.

9. A pharmaceutical composition for promoting hair follicle regeneration, comprising:
dermal papilla cells (DP cells) aggregating a sphere; and
adipocyte lineage cells enclosing the sphere.

10. The pharmaceutical composition of claim 9, wherein the adipocyte lineage cells are human adipocytes, human adipose-derived stem cells (ASCs), or a combination thereof.

11. The pharmaceutical composition of claim 9, wherein the DP cells and the adipocyte lineage cells are in a ratio of 2 to 1.

12. A pharmaceutical composition for promoting hair follicle regeneration, comprising:
dermal papilla cells (DP cells); and
adipocyte lineage cells;
wherein the DP cells and the adipocyte lineage cells jointly assemble a mixed sphere.

13. The pharmaceutical composition of claim 12, wherein the adipocyte lineage cells are human adipocytes, human adipose-derived stem cells (ASCs), or a combination thereof.

14. The pharmaceutical composition of claim 12, wherein the DP cells and the adipocyte lineage cells are in a ratio of 2 to 1.
